(19) United States
(12) Patent Application Publication
(10) Pub. No.: US 2010/0312130 A1
(43) Pub. Date: Dec. 9, 2010

(54) GRADED RESPONSE TO MYOCARDIAL ISCHEMIA

(76) Inventors: Yi Zhang, Plymouth, MN (US); Kent Lee, Shoreview, MN (US); Shibaji Shome, Minneapolis, MN (US); Qi An, New Brighton, MN (US); Ramesh Wariar, Blaine, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/860,152

(22) Filed: Aug. 20, 2010

Related U.S. Application Data
(63) Continuation-in-part of application No. 11/426,835, filed on Jun. 27, 2006.

(51) Int. Cl. A61B 5/02

(52) U.S. Cl. 600/508

(57) ABSTRACT

Severity and confidence level of a myocardial ischemia event can be determined, such as including using an ambulatory medical device, and such information can be used to determine a graded response to the myocardial ischemia event.

### CLAIM OF PRIORITY

This application is a continuation-in-part of, and claims the benefit of priority under 35 U.S.C. §120 to, U.S. patent application Ser. No. 11/426,835, entitled “Detection Of Myocardial Ischemia From The Time Sequence Of Implanted Sensor Measurements,” filed on Jun. 27, 2006, published as US 2007/0299356 on Dec. 27, 2007, which is herein incorporated by reference in its entirety.

### BACKGROUND

Cardiac rhythm management devices can include implantable or other ambulatory devices, such as pacemakers, cardioverter defibrillators, or devices that can provide a combination of pacing, defibrillation, cardiac resynchronization therapy, for cardiovascular monitoring, or the like. In an example, such devices can be used to detect or treat heart failure. In an example, such devices can be used to detect or treat episodes of myocardial ischemia. Myocardial ischemia is a condition caused by a reduced blood supply to the myocardial tissue of the heart. Stadler et al. U.S. Patent Publication No. 2004/0122478, entitled METHOD AND APPARATUS FOR GAUGING SEVERITY OF MYOCARDIAL ISCHEMIC EPISODES, refers to an implantable medical device and method for detecting ischemia in a human heart, determining a severity of ischemia, and providing a response from the implantable medical device to the patient. (See Stadler et al. at Abstract.) Wariar et al. U.S. Patent Publication No. US 2007/0299356, entitled DETECTION OF MYOCARDIAL ISCHEMIA FROM THE TIME SEQUENCE OF IMPLANTED SENSOR MEASUREMENTS, refers to the detection of myocardial ischemia using a system including a plurality of implantable sensors, a processor, and a response circuit.

### OVERVIEW

This document describes, among other things, an apparatus and method in which a confidence level of a myocardial ischemia event having occurred can be determined and used to determine a graded response to the myocardial ischemia event.

Example 1 includes subject matter that can include an apparatus comprising: an ambulatory medical device, including a ischemia detector circuit configured to detect an indication of a myocardial ischemia event; and a processor circuit, configured to be communicatively coupled to the ischemia detector and to: receive the indication of the myocardial ischemia event; determine a confidence level of the myocardial ischemia event having occurred; and respond using the confidence level, the responding including at least one of initiating, selecting, or adjusting a response.

In Example 2, the subject matter of Example 1 can optionally include the processor circuit configured to determine a severity indicator value of the indication of the myocardial ischemia event.

In Example 3, the subject matter of one or any combination of Examples 1-2 can optionally include the processor circuit configured such that the responding comprises responding using both the severity indicator value and the confidence level.

In Example 4, the subject matter of one or any combination of Examples 1-3 can optionally include the processor circuit configured such that the severity indicator is multi-valued and the confidence level is multi-valued.

In Example 5, the subject matter of one or any combination of Examples 1-4 can optionally include the processor circuit configured such that the determining the confidence level comprises using a regression model.

In Example 6, the subject matter of one or any combination of Examples 1-5 can optionally include the processor circuit configured such that the detecting the indication of the myocardial ischemia event comprises using one or more sensor measurements from the ischemia detector circuit to detect the indication of the myocardial ischemia event; and the determining the confidence level comprises computing a probability according to:

$$P = \frac{1}{1 + {e}^{- z}} ,$$

wherein z=b₀+b₁X₁+b₂X₂+ . . . +bₘXₘ, P is a probability of a myocardial ischemia event occurring, z is a measure of a total contribution of all of the one or more sensor measurements used, b₀ is a logistic regression intercept, and b₁, b₂, . . . bₘ are the logistic regression coefficients of the one or more sensor measurements X₁, X₂, . . . Xₘ respectively.

In Example 7, the subject matter of one or any combination of Examples 1-6 can optionally include the ambulatory medical device comprising an implantable medical device including the processor circuit.

In Example 8, the subject matter of one or any combination of Examples 1-7 can optionally include the processor circuit configured such that the confidence level is determined using a time-wise sequence of multiple indications of the myocardial ischemia event.

Example 9 can include, or can optionally be combined with the subject matter of one or any combination of Examples 1-8 to include subject matter (such as a method, a means for performing acts, or a machine-readable medium including instructions that, when performed by the machine, cause the machine to perform acts), comprising: detecting an indication of a myocardial ischemia event; determining a confidence level of the myocardial ischemia event having occurred; and responding using the confidence level, the responding including at least one of initiating, selecting, or adjusting a response.

In Example 10, the subject matter of one or any combination of Examples 1-9 can optionally include instructions that, when performed by the device, comprise determining a severity indicator value of the indication of the myocardial ischemia event.

In Example 11, the subject matter of one or any combination of Examples 1-10 can optionally include instructions that, when performed by the device, comprise responding using both the severity indicator value and the confidence level.

In Example 12, the subject matter of one or any combination of Examples 1-11 can optionally include instructions such that, when performed by the device, the severity indicator is multi-valued and the confidence level is multi-valued.

In Example 13, the subject matter of one or any combination of Examples 1-12 can optionally include instructions such that, when performed by the device, the determining the confidence level comprises using a regression model.

In Example 14, the subject matter of one or any combination of Examples 1-13 can optionally include instructions such that, when performed by the device, the detecting the indication of the myocardial ischemia event comprises using one or more sensor measurements to detect the indication of the myocardial ischemia event; and the determining the confidence level comprises computing a probability according to:

$$P = \frac{1}{1 + {e}^{- z}} ,$$

wherein z=b₀+b₁X₁+b₂X₂+ . . . +bₘXₘ, P is a probability of a myocardial ischemia event occurring, z is a measure of a total contribution of all of the one or more sensor measurements used, b₀ is a logistic regression intercept, and b₁, b₂, . . . bₘ are the logistic regression coefficients of the one or more sensor measurements X₁, X₂, . . . Xₘ respectively.

In Example 15, the subject matter of one or any combination of Examples 1-14 can optionally include instructions such that, when performed by the device, the responding comprises providing a local alert.

In Example 16, the subject matter of one or any combination of Examples 1-15 can optionally include instructions such that, when performed by the device, the determining the confidence level comprises using a time-wise sequence of multiple indications of the myocardial ischemia event.

Example 17 can include, or can optionally be combined with the subject matter of one or any combination of Examples 1-16 to include subject matter (such as a method, a means for performing acts, or a machine-readable medium including instructions that, when performed by the machine, cause the machine to perform acts) comprising: detecting an indication of a myocardial ischemia event using an ambulatory medical device; determining a confidence level of the myocardial ischemia event having occurred; and responding using the confidence level, the responding including at least one of initiating, selecting, or adjusting a response.

In Example 18, the subject matter of one or any combination of Examples 1-17 can optionally be performed comprising determining a severity indicator value of the indication of the myocardial ischemia event.

In Example 19, the subject matter of Examples 1-18 can optionally be performed such that the responding comprises responding using both the severity indicator value and the confidence level.

In Example 20, the subject matter of Examples 1-19 can optionally be performed such that the severity indicator is multi-valued and the confidence level is multi-valued.

These examples can be combined in any permutation or combination. This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of portions of an apparatus that can enable detecting and responding to a myocardial ischemia event.

FIG. 2 illustrates an example of portions of an implanted medical device.

FIG. 3 illustrates an example of portions of a remote interface that can enable detecting and responding to a myocardial ischemia event.

FIG. 4 illustrates an example of a technique for providing a graded response to an indication of a myocardial ischemia event.

FIG. 5 illustrates an example of a relationship between a confidence level of a myocardial ischemia event having occurred, a severity indicator value of the myocardial ischemia event, and a graded response to the myocardial ischemia event.

### DETAILED DESCRIPTION

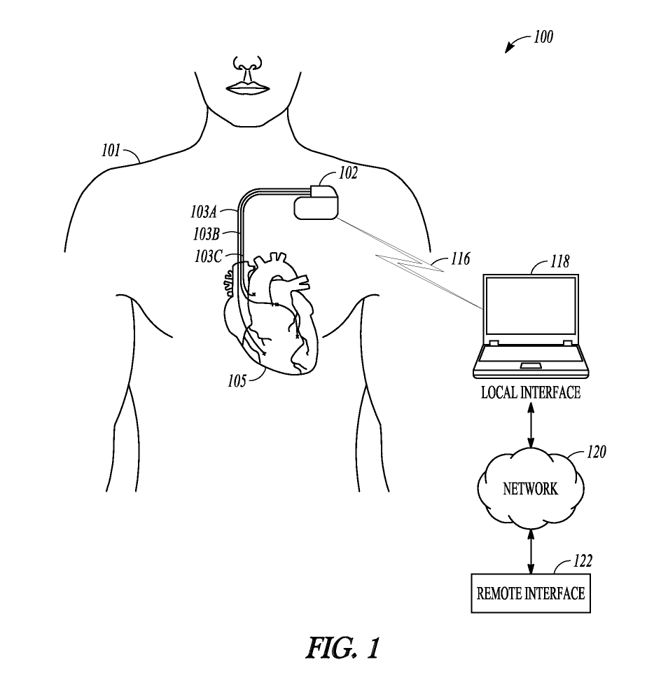

FIG. 1 illustrates an example of portions of an apparatus 100 that can enable detecting and responding to a myocardial ischemia event. In the example of FIG. 1, an ambulatory medical device, such as an implantable medical device (IMD) 102 can be configured to monitor or provide therapy to a patient 101. In an example, an ambulatory medical device can include an external (e.g., wearable) medical device or an implantable medical device, among one or more other devices. For example, ambulatory medical devices can include one or more of a pacemaker, an implantable cardioverter defibrillator (ICD), a cardiac resynchronization therapy pacemaker (CRT-P), a cardiac resynchronization therapy defibrillator (CRT-D), a pulmonary artery (PA) pressure sensor, a neurostimulation device, a physiological signals monitor, a cardiovascular monitor, a stent, a drug pump, or the like. In an example, the IMD 102 can be configured to sense physiological data, derive physiological measures or correlations, or store data such as for later communication or reference. Examples of physiological data can include implantable electrograms, surface electrocardiograms, heart rate intervals (e.g., AA, VV, AV or VA intervals), electrogram templates such as for tachyarrhythmia discrimination, pressure (e.g., intracardiac or arterial pressure), oxygen saturation, activity, heart rate variability, heart sounds, impedance, respiration, posture, intrinsic depolarization amplitude, or the like. While only one IMD 102 is illustrated in FIG. 1, more than one IMD 102 may be implanted. For example, medical devices that have specific functions can be placed in accordance with their function. In addition, the IMD 102 can be composed of more than one device, with each device having one or more functions. Similarly, the position of the IMD 102 can vary. Examples of other locations can include the patient's abdomen, back, arm, or the like.

In an example, the IMD 102 can include one or more intracardiac leads 103A-C, implanted in a human body with portions of the intracardiac leads 103A-C inserted into the heart 105. The intracardiac leads 103A-C can include one or more electrodes, positionable within the heart 105, configured to sense electrical activity of the heart 105, or to deliver electrical stimulation energy to the heart 105. In an example, one or more of the intracardiac leads 103A-C can be configured to deliver pacing pulses to treat various arrhythmias. In an example, one or more of the intracardiac leads 103A-C can be configured to deliver pacing pulses or defibrillation shocks, such as to treat one or various arrhythmias. In an example, the IMD 102 can include one or more extracardiac leads (not illustrated on FIG. 1), such as subcutaneous leads, sub-pectoral leads, and epicardial leads.

In an example, the IMD 102 can be configured to be capable of bidirectional communication using a connection 116 with an external or other local interface 118. Examples of the connection 116 can include radio frequency (RF), blue tooth, infrared, or one or more other communication connections. A local interface 118 can be a device configured such as to receive input, process instructions, store data, present data in a human-readable form, or communicate with other devices. The IMD 102 can be configured to receive commands from the local interface 118 or to communicate one or more patient indications to the local interface 118. Examples of patient indications can include one or more sensed or derived measurements such as heart rate, heart rate variability, data related to ischemia events, data related to tachyarrhythmia episodes, hemodynamics and hemodynamic stability, respiration, cardiac motion, cardiac contractility, activity, therapy history, autonomic balance, motor trends, electrogram templates for tachy discrimination, heart rate variability trends or templates, or trends, templates, or abstractions derived from sensed physiological data. Patient indications can include or be derived from one or more physiological indications, such as the physiological data described above. The IMD 102 can also be configured to communicate one or more device indications to the local interface 118. Examples of device indications can include lead/shock impedance, pacing amplitudes, pacing capture thresholds, or one or more other device metrics. In an example, the IMD 102 can be configured to communicate sensed physiological signal data to the local interface 118, which can then communicate the signal data to a remote device such as for processing. In an example, when more than one IMD 102 has been employed, the multiple IMD 102 devices can be configured to communicate with other, such as by using the connection 116.

In an example, the local interface 118 can be located in close proximity to the patient 101. The local interface 118 can be attached, coupled, integrated or incorporated with a personal computer or a specialized device, such as a medical device programmer. In an example, the local interface 118 can be a hand-held device, such as a personal digital assistant (PDA) or smart phone. In examples, the local interface 118 can be a specialized device or a personal computer. In an example, the local interface 118 can be adapted to communicate with a remote interface 122. Examples of a remote interface include a remote computer or server or the like. The communication link between the local interface 118 and the remote interface 122 can be made through a computer or telecommunications network 120. The network 120 can include, in various examples, one or more wired or wireless networking such as the Internet, satellite telemetry, cellular or other mobile telephone telemetry, microwave telemetry, or using one or more other long-range communication networks.

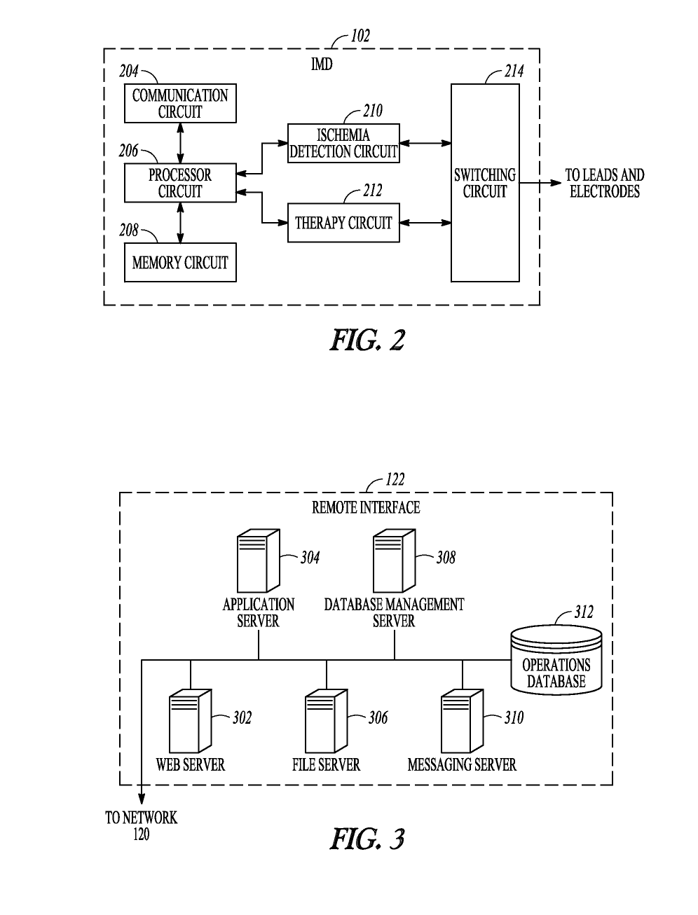

FIG. 2 illustrates an example of portions of the IMD 102. In the example of FIG. 2, the IMD 102 can include a switching circuit 214, such as for selectively connecting to one or more of the various sensors, such as can be located on the leads 103A-C or elsewhere. In an example, an ischemia detector circuit 210 can be selectively coupled to various sensors, such as by the switching circuit 214. In an example, the ischemia detector circuit 210 can include one or more sense amplifiers, filter circuits, or other circuits such as for sensing or signal-processing one or more signals, such as cardiac signals. In an example, the ischemia detector circuit 210 can be configured to detect an indication of a myocardial ischemia event using one or more physiologic sensors, such as explained further below. Examples of sensors that can be used to detect the indication of the myocardial ischemia event can include, but are not limited to, one or more of: an electrical cardiac signal sensing circuit, a heart sounds sensor, a transthoracic impedance measurement circuit, an intracardiac impedance measurement circuit, an accelerometer, a blood pressure sensor, a wall motion sensor, a heart rate variability sensor, or a physical activity sensor. In an example, a therapy circuit 212 can be selectively coupled to various sensors, such as by the switching circuit 214. In an example, the therapy circuit 212 can include therapy energy generation circuitry (e.g., capacitive, inductive, or other) such as for generating, storing, or delivering an electrostimulation, cardioversion, defibrillation, drug delivery, or other energy.

In an example, the ischemia detector circuit 210 or the therapy circuit 212 can be coupled to a processor circuit 206. The processor circuit 206 can perform instructions, such as for signal processing of signals derived by the ischemia detector circuit 210, or for controlling operation of the therapy circuit 212, or for controlling one or more other operations of the IMD 102. In an example, the processor circuit 206 can be configured to determine a severity indicator value, such as by using the indication of the myocardial ischemia event received from the ischemia detector circuit 210, to determine a confidence level of the myocardial ischemia event having occurred, and to respond using the confidence level, such as explained further below. In an example, the processor circuit 206 can be coupled to or include a memory circuit 208, such as for storing or retrieving instructions or data. The processor circuit 206 can be coupled to or include a communication circuit 204, such as for communicating with another location, such as with the local interface 118.

In an example, the IMD 102 can include multiple processor circuits 206. One or more processor circuits can be included in one or more of the IMD 102, the local interface 118, or the remote interface 122, such as for distributing the processing load, such as for decreasing the power consumption of the IMD 102.

FIG. 3 illustrates an example of the remote interface 122. In an example, the remote interface 122 can include one or more computers, such as a database management server 308, a messaging server 310, a file server 306, an application server 304, or a web server 302. The database management server 308 can be configured to provide one or more database services to one or more clients, which can include one or more other servers, such as in the remote interface 122. The messaging server 310 can be configured to provide a communication platform for one or more users of the remote interface 122. For example, the messaging server 310 can provide an email communication platform. Examples of other types of messaging can include one or more of short message service (SMS), instant messaging, or paging services. The file server 306 can be used to store patient data, device data, documents, images, and other files for the web server 302 or as a general document repository. The application server 304 can provide one or more applications to the web server 302. To enable some of these services provided by these servers 302, 304, 306, 308, and 310, the remote interface 122 can include an operations database 312. The operations database 312 can be used for various functions and can be composed of one or more logically or physically distinct databases. The operations database 312 can be used to store clinical data such as for individual patients, one or more patient populations, one or more patient trials, or the like. In an example, the operations database 312 can be used to store patient data such as for individual patients, one or more patient populations, one or more patient trials, or the like. For example, the operations database 312 can include a copy of, a portion of, a summary of, or other data from an electronic medical records (EMR) system. In an example, the operations database 312 can store device information, such as one or more device settings such as for a particular patient or a group of patients, one or more preferred device settings such as for a particular clinician or a group of clinicians, device manufacturer information, or the like. In an example, the operations database 312 can be used to store raw, intermediate, or summary data such as of one or more patient indications, for example, along with one or more probabilistic outcomes (e.g., a patient population profile and a corresponding 1-year survival curve).

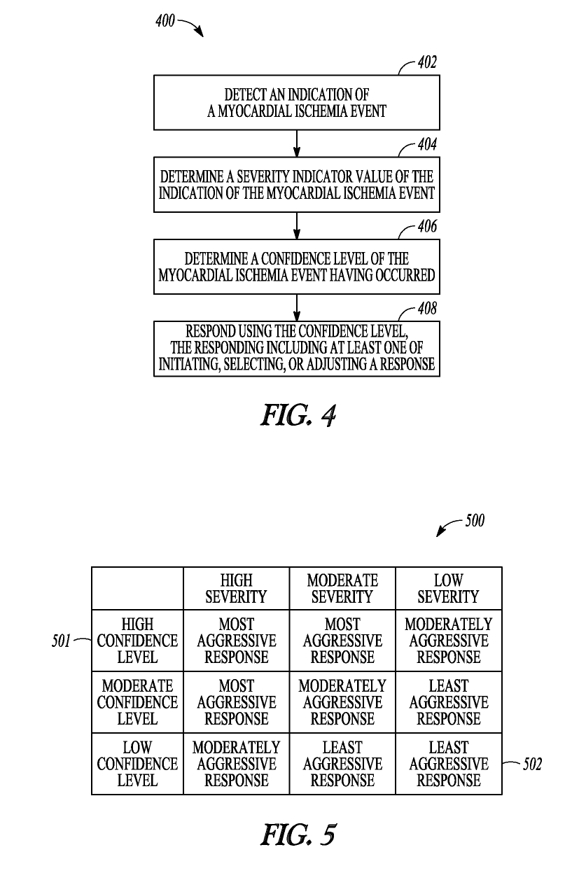

FIG. 4 is a diagram illustrating an example of a technique 400 for providing a graded response to a detected indication of a myocardial ischemia event. At 402, an indication of a myocardial ischemia event can be detected. In an example, the myocardial ischemia event can be detected using an electrocardiogram (ECG) signal of the electrical activity of the heart 105. In an example, the myocardial ischemia event can be detected using the ST segment of the ECG, such as by comparing the ST segment of the ECG to that of a baseline ECG such as to detect an ST segment deviation that can be indicative of a myocardial ischemia event. In an example, the myocardial ischemia event can be detected using the QRS complex of the ECG, such as by comparing the width or morphology of the detected QRS complex to that of a baseline ECG. For example, a shortening of the QT interval can indicate an increased likelihood of a myocardial ischemia event, and the indication can be adjusted accordingly. In an example, the myocardial ischemia event can be detected by sensing the mechanical activity of the heart, such as by using an accelerometer or other heart sounds sensor to sense one or more signals indicative of regional cardiac wall motion. A regional shortening of a heart wall can indicate an increased likelihood of a myocardial ischemia event. In such case, the indication of the myocardial ischemia event can be adjusted to indicate an increased likelihood of the myocardial ischemia event. In an example, the myocardial ischemia event can be detected using the patient's heart rate, such as by using a heart rate sensor to detect an increase in the heart rate of a patient. An increase in heart rate in the absence of exercise can indicate an increased likelihood of a myocardial ischemia event. Some patients may experience about a forty percent increase in heart rate during a myocardial ischemia event. The indication of the myocardial ischemia event can be adjusted to indicate an increased likelihood of the event if the patient's heart rate increases in the absence of exercise by an amount greater than a threshold value. In an example, the myocardial ischemia event can be detected using a change in heart chamber contractility or relaxation, such as by using a cardiac impedance sensor to detect the rate of change of intra-chamber blood pressure (dP/dt). During a myocardial ischemia event, some patients may experience a decrease in heart chamber relaxation as measured by a change in absolute value of a maximum negative dP/dt of up to forty percent. Some subjects may experience a decrease in heart chamber contractility during a myocardial ischemia event, as measured by a change in maximum positive dP/dt of up to twenty percent. In such case, the indication can be adjusted to indicate an increased likelihood of the event.

In an example, multiple sensors can be used to detect multiple indications of a myocardial ischemia event. For example, multiple sensors, such as those described above, can be used to detect multiple indications of the myocardial ischemia event.

At 404, a severity indicator value of the indication of the myocardial ischemia event can be determined. In an example, a duration of the indication of the myocardial ischemia event can be used as a factor to determine the severity indicator value. For example, a more severe myocardial ischemia indicator value can be assigned to an indication of a myocardial ischemia event that is longer in duration than to an indication of a myocardial ischemia event that is shorter in duration. For example, an ST segment elevation that is longer in duration can be assigned a severity indicator value that is more severe than an ST segment elevation that is shorter in duration.

In an example, a location of an indication of the myocardial ischemia event can be used as a factor to determine the severity indicator value. For example, a more severe severity indicator value can be assigned to an indication of a myocardial ischemia event that occurs more proximal of a coronary artery than to an indication of a myocardial ischemia event that occurs more distal of the coronary artery. In some cases, a patient can experience frequent myocardial ischemia events. In such cases, there can be a significant burden on the patient. To address the potential burden on the patient that can be caused by frequent myocardial ischemia events, a myocardial ischemia event can be characterized as more severe if the frequency of the myocardial ischemia events is higher. In an example, the frequency of occurrence of indications of myocardial ischemia events can be used as a factor to determine the severity indicator value. This can include assigning a more severe severity indicator value to an indication of a myocardial ischemia event if the frequency of occurrence of indications of myocardial ischemia events is higher than if the frequency of occurrence of indications of myocardial ischemia events is lower.

In an example, a rate of change with respect to time of the indication of the myocardial ischemia event can be used as a factor to determine the severity indicator value. This can include assigning a more severe severity indicator value to an indication of a myocardial ischemia event if the rate of change of the indication of the myocardial ischemia event is greater in magnitude than if the rate of change is lesser in magnitude.

In an example, the severity indicator value can be determined using one or more of the duration, location, frequency of occurrence, or rate of change of the indication of the myocardial ischemia event. For example, multiple individual severity indicator values can be assigned to the indication of the myocardial ischemia event using one or more of the duration, the location, the frequency of occurrence, or the rate of change of the indication of the myocardial ischemia event. These multiple individual severity indicator values can then be used to determine an overall severity indicator value, such as by assigning an overall or combined severity indicator value using a mean, median, mode or other central tendency of the multiple individual severity indicator values.

In an example, one or more of the multiple severity indicator values can be used to trigger the use of one or more severity indicator values in the determination of the overall severity indicator value. For example, the severity indicator value assigned using the rate of change of the event can be used in the determination of the overall severity indicator value of the indication of the myocardial ischemia event if the severity indicator value assigned using the location of the event exceeds a threshold value.

In an example, the severity indicator value can be multi-valued. For example, the severity indicator value can be represented by three discrete values respectively representing a high-severity myocardial ischemia event, a moderate-severity myocardial ischemia event, or a low-severity myocardial ischemia event. There exist many possible configurations in which the severity indicator value can be multi-valued. The above illustrative example is but one possible configuration.

In an example, the severity indicator value can be determined such as by comparing the indication of the myocardial ischemia event to a threshold value. For example, the severity indicator value can be determined such as by comparing one or more of the duration, location, frequency of occurrence, or rate of change of the indication of the myocardial ischemia event to a threshold value. In an example, the severity indicator value of the indication of the myocardial ischemia event can be assigned a high-severity severity indicator value if the indication of the myocardial ischemia event is greater than the threshold value, and can be assigned a low-severity severity indicator value if the indication of the myocardial ischemia event is less than the threshold value. In an example, the specified threshold value can be multi-valued. For example, the multi-valued severity indicator value can be determined such as by comparing the indication of the myocardial ischemia event to the multi-valued threshold value.

In an example, multiple indications of myocardial ischemia events or the severity indicator values assigned to the corresponding myocardial ischemia indications can be obtained over time and represented, such as using trending over time or using one or more histograms. In an example, the severity indicator value of an indication of a myocardial ischemia event can be determined using the relative position of the indication of the myocardial ischemia event in the histogram.

At 406, a confidence level of the occurrence of the myocardial ischemia event can be determined. In an example, the confidence level can be determined using one or more regression models. A regression model can relate one or more response variables to one or more predictor variables. A regression model can be expressed as:

y=ƒ(x,β)+ε

where y represents the one or more response variables, x represents the one or more predictor variables, β represents one or more unknown model parameters, and ε represents a noise term. Examples of regression models can include, but are not limited to: linear regression models, logistic regression models, artificial neural networks, or decision trees.

In an example, the confidence level of the myocardial ischemia event having occurred can be determined such as by using a linear regression model, which can be expressed as:

y=β₀+β₁x₁+ . . . +βₖxₖ+ε,

where βᵢ, i=0, . . . k represents the model parameters that determine the relative contribution of predictor variables xᵢ, i=1, . . . k. The model parameters βᵢ, i=0, . . . k can be determined from a training set by estimation methods, such as a least squares method, a least absolute deviation method, or a maximum likelihood method.

In an example, the confidence level of the myocardial ischemia event having occurred can be determined such as by using a logistic regression model, which can be expressed as:

$$P = \frac{1}{1 + {e}^{- z}} ,$$

where z=b₀+b₁X₁+b₂X₂+ . . . +bₘXₘ, P is a probability of the myocardial ischemia event having occurred, z is a measure of a total contribution of all of the one or more sensor measurements used, b₀ is a logistic regression intercept and b₁, b₂, . . . bₘ are the logistic regression coefficients of the one or more sensor measurements X₁, X₂, . . . Xₘ respectively. The confidence level of the myocardial ischemia event having occurred can be increased such as by selecting the number or type of the one or more sensor measurements used by the logistic regression model.

In an example, the confidence level of the myocardial ischemia event having occurred can be increased at least in part by selecting the one or more sensor measurements to be used by the logistic regression model. For example, the probability P of the myocardial ischemia event having occurred that is produced by the logistic regression model in a particular configuration can be compared to a reference myocardial ischemia event that is known to have a very high likelihood of occurrence. The contribution of a sensor measurement to the probability P of the logistic regression model can be determined by comparing the calculated probability P to the likelihood of occurrence of the reference myocardial ischemia event.

In an example, the one or more sensors used by the logistic regression model can be selected at least in part using a forward selection technique. In such an example, the contribution of sensor measurements from individual sensors can be added to the logistic regression model one at a time. In an example, a particular sensor measurement can be selected such as by the user to be used by the logistic regression model if it changes the calculated probability P by an amount that meets or exceeds a statistically significant threshold value.

In an example, the one or more sensor measurements used by the logistic regression model can be selected at least in part using a backward selection method. In such an example, the probability P can first be calculated by the logistic regression model using the available set of sensor measurements. Next, the contribution of sensor measurements from individual sensors can be removed from the logistic regression model until the probability P calculated by the logistic regression model diverges by a threshold amount from the likelihood of occurrence of the reference myocardial ischemia event. For example, a threshold value T can be chosen, such as by the user. The probability P can first be calculated by the logistic regression model using measurements from an available set of sensors, such as an implantable electrogram, a heart sounds sensor, an intracardiac pressure sensor, a cardiac impedance sensor, and a heart rate sensor. Next, the probability P can be calculated without the contribution of sensor measurements from the cardiac impedance sensor, using only the heart sounds sensor, the intracardiac pressure sensor, and the heart rate sensor. The probability P can be compared to the likelihood of occurrence of the reference myocardial ischemia event. If, after removing the contribution of this sensor measurement from the cardiac impedance sensor, the calculated probability P does not differ from the likelihood of occurrence of the reference myocardial ischemia event by an amount greater than the threshold value T, the contribution of this sensor measurement from the cardiac impedance sensor can be removed from the logistic regression model. The backward selection method can continue until, upon removal of the contribution of a sensor measurement from an individual sensor, the calculated probability P differs from the likelihood of occurrence of the reference myocardial ischemia event by an amount greater than the threshold value T.

In an example, the one or more sensor measurements used by the logistic regression model can be selected at least in part using a stepwise regression method. In such an example, the contribution of a sensor measurement from an individual sensor can be added to the logistic regression model one at a time. A particular sensor measurement can be selected to be used by the logistic regression model if it changes the calculated probability P by an amount that meets or exceeds a statistically significant threshold value. Next, the contribution of sensor measurements from any previously selected sensor measurements can be rechecked, such as by removing a particular previously selected sensor measurement from the logistic regression model. If the calculated probability P does not change by a statistically significant threshold value after the removal of the contribution of the particular previously selected sensor measurement, then that particular previously selected sensor measurement can be removed from the logistic regression model.

In an example, the one or more sensors used by the logistic regression model can be selected at least in part using a hierarchical regression method. In such an example, the one or more sensors can be grouped according to the one or more characteristics of the data they detect (e.g. hemodynamic function, ECG characteristics, or mechanical function) and added to or removed from the logistic model as a group.

In an example, the one or more sensors used by the logistic regression model can be selected at least in part using one or more of the forward selection, backward selection, stepwise regression, or hierarchical regression techniques described above. For example, one or more temporary sets of sensors to be used by the logistic regression model can be selected using one or more of the previously described techniques. Those sensors that appear in more than one of the temporary sets of temporary sensors can be selected for use by the logistic regression model.

In an example, the confidence level of the myocardial ischemia event having occurred can be determined at least in part using an artificial neural network. An artificial neural network can include a mathematical model that can be used to determine a relationship between one or more response variables and one or more predictor variables. For example, an artificial neural network can be used at least in part to model the function ƒ in the regression model described above.

In an example, the confidence level of the myocardial ischemia event having occurred can be determined at least in part using a decision tree. A decision tree can be used to infer the probability of an outcome of an event, such as by using one or more decision nodes, chance nodes, and end nodes to calculate the probability of the occurrence of an end node using the state and probability of the decision and chance nodes.

In an example, the time of day of the indication of the myocardial ischemia event can be used to determine the confidence level of the myocardial ischemia event having occurred. For example, this can include assigning a higher confidence level to an indication of a myocardial ischemia event that occurs during morning hours than to an indication of a myocardial ischemia event that occurs during non-morning hours.

In an example, the relative differences in one or more characteristics of the type of data detected by the one or more sensors can be used to determine the confidence level of the myocardial ischemia event having occurred. In such an example, a higher confidence level can be assigned to the indication of the myocardial ischemia event if the indication was provided by at least two sensors of fundamentally different type (e.g. hemodynamic function, ECG characteristics, mechanical function) than if the indication was provided by at least two sensors of the same type.

In an example, the confidence level can be multi-valued. For example, the confidence level can be represented by three discrete values representative of a high confidence level, a moderate confidence level, or a low confidence level of the indication of the myocardial ischemia event having occurred. There exist many possible configurations in which the confidence level can be multi-valued and the example mentioned above is but one possible configuration. In an example, the confidence level can be continuous.

In an example, one or more of the regression models, time-wise sequence of indications, time of day, or relative differences in the type of sensor described above can be used to determine the confidence level of the myocardial ischemia event having occurred. For example, multiple confidence levels can be determined using the techniques described above. The multiple confidence levels can be used to determine a combined confidence level, such as by assigning the combined confidence level using a mean, median, or other central tendency value of the multiple confidence levels.

At 408, a response can be initiated, selected, or adjusted at least in part by using the confidence level. In an example, this can be done by one or more of the IMD 102, the local interface 118, the remote interface 122, or elsewhere. Examples of responses can include, but are not limited to: providing a local alert, providing a remote alert, delivering anticoagulant therapy, or starting anti-arrhythmic treatment.

In an example, the response can include providing a local alert, such as to the patient 101, such as by providing a physiological stimulation, such as a vibration, to the patient 101 to alert the patient 101 of the occurrence of the indication of the myocardial ischemia event. In an example, the response can include sending one or more of an email or telephone message to alert one or more of the patient, a caregiver, a clinician, or another of the occurrence of the indication of the myocardial ischemia event. In an example, the response can include sending a message using one or more of a short messaging service (SMS), instant messaging service, or paging service. In an example, the response can include one or more of creating, amending, or saving a file such as by using one or more of the IMD 102, the local interface 118, the remote interface 122, or others. In such an example, the file can include a copy of, a portion of, a summary of, or other data relating to the one or more indications of the myocardial ischemia event, or its severity, or its confidence level. In an example, the response can include sending a file to a web server. In an example, the response can include one or more of initiating, selecting, or adjusting one or more of an audible alert, textual alert, or graphical image.

In an example, the response can be graded. For example, available responses can be grouped into categories such as by determining a level of aggressiveness of each available response. In an example, the response can be categorized such as by three discrete grades representing a most-aggressive response, a moderately-aggressive response, or a least-aggressive response. In such an example, each of the available responses can be categorized into one of the grades.

In an example, a least-aggressive response can include one or more of creating or saving a summary file of the indication of the myocardial ischemia event for later review. In an example, a least-aggressive response can include initiating one or more of an email or SMS message such as to the patient 101 or to a caregiver.

In an example, a moderately-aggressive response can include initiating an alert to a nurse, such as by providing to a local or remote interface one or more of an audible, textual, or graphical alert. In an example, a moderately-aggressive response can include adjusting a local or remote alert, such as by adjusting one or more of an audible, textual, or graphical alert such as to indicate a moderate confidence level of an indication of a myocardial ischemia event. In an example, a moderately-aggressive response can include sending an email with a high-priority status.

In an example, a most-aggressive response can include initiating an alert to a physician, such as by providing to a local or remote interface one or more of an audible, textual, or graphical alert. In an example, a most-aggressive response can include initiating a telephonic message to one or more of the patient 101 or a physician. In an example, a most-aggressive response can include initiating an anticoagulant therapy, such as by using an ambulatory drug pump. In an example, a most-aggressive response can include initiating an anti-arrhythmic treatment, such as by using an implantable cardioverter defibrillator. In an example, a most-aggressive response can include adjusting one or more of a local or remote alert, such as by adjusting one or more of an audible, textual, or graphical alert such as to indicate a high confidence level of an indication of a myocardial ischemia event.

Table 1 illustrates an example of a configuration of graded responses.

TABLE 1

| Most-Aggressive | Moderately-Aggressive | Least Aggressive |
|---|---|---|
| Response | Response | Response |
| Alerting a physician | Alerting a nurse | Creating or saving |
| | | a summary file |

However, there exist many possible configurations in which the responses can be characterized. Factors to consider in grading a response can include, but are not limited to: a preferred method of communication, a patient's prior health history, an availability of one or more communication channels, an availability of one or more caregivers, or an availability of different types of caregivers.

Table 2 illustrates another example of a configuration of graded responses.

TABLE 2

| Most-Aggressive | Moderately-Aggressive | Least Aggressive |
|---|---|---|
| Response | Response | Response |
| Alerting a physician | Alerting a nurse | Creating or saving |
| | | a summary file |
| Initiating a | Adjusting an alert to | Adjusting an alert |
| telephonic message to | indicate a moderate | to indicate a low |
| a physician. | confidence level | confidence level |
| Initiating | Sending an email with | Sending an email |
| anticoagulant therapy | high-priority status | or SMS message to |
| or anti-arrhythmic | to a caregiver | the patient 101 |
| treatment | | |

In the example of Table 2, one or more of the responses from the particular grade of responses can be initiated. In an example, the one or more of the graded responses can be cumulative. For example, a graded response can include those responses from lower grades. Table 3 illustrates an example of graded responses including one or more cumulative responses.

TABLE 3

| Most-Aggressive | Moderately-Aggressive | Least Aggressive |
|---|---|---|
| Response | Response | Response |
| Alerting a physician | Alerting a nurse and | Creating or saving |
| and alerting a nurse | creating or saving a | a summary file |
| and creating or | summary file | |
| saving a summary file | | |
| Initiating a | Adjusting an alert to | Adjusting an alert |
| telephonic message to | indicate a moderate | to indicate a low |
| a physician and | confidence level | confidence level |
| adjusting an alert | | |
| to indicate a high | | |
| confidence level | | |
| Initiating | Sending an email with | Sending an email |
| anticoagulant therapy | high-priority status | or SMS message to |
| or anti-arrhythmic | to a caregiver and | the patient 101 |
| treatment and sending | sending an email or | |
| an email with | SMS message to the | |
| high-priority status | patient 101 | |
| to a caregiver and | | |
| sending an email or | | |
| SMS message to the | | |
| patient 101 | | |

In an example, the response can be initiated, selected, or adjusted by one or more of the IMD 102, the local interface 118, or the remote interface 122, at least in part by using the confidence level of the indication of the myocardial ischemia event. For example, a highly-aggressive response can be provided if the confidence level of the indication of the myocardial ischemia event is high. In an example, a moderately-aggressive response can be provided if the confidence level is moderate. In an example, a least-aggressive response can be provided if the confidence level is low.

In an example, the response can be initiated, selected, or adjusted by one or more of the patient 101, the caregiver, or the clinician. In an example, the response can be initiated, selected, or adjusted for the patient 101. In an example, the response can be initiated, selected, or adjusted for a group of patients 101.

In an example, the response can be initiated, selected, or adjusted using both the confidence level of the myocardial ischemia event having occurred and also the severity indicator value of the indication of the myocardial ischemia event. FIG. 5 illustrates an example 500 of a relationship between the confidence level, the severity indicator value, and the graded response to the indication of the myocardial ischemia event. At 501, both the severity indicator value and the confidence level of the myocardial ischemia event having occurred are high. In the example of FIG. 5, the most aggressive response can be initiated, selected, or adjusted, such as at 501. In an example, as in the example of FIG. 5, the most aggressive response can be initiated, selected, or adjusted such as when neither the confidence level nor the severity indicator value are at their highest value. For example, the most aggressive response can be initiated, selected, or adjusted such as when the confidence level is moderate and the severity indicator value is high. In such an example, it can be desirable to respond using the most aggressive response even though the confidence level is not high because of the high severity indicator value of the myocardial ischemia event.

At 502, both the severity indicator value and the confidence level of the myocardial ischemia event having occurred are low. In the example of FIG. 5, the least aggressive response can be initiated, selected, or adjusted, such as at 502. In an example, as in the example of FIG. 5, the least aggressive response can be initiated, selected, or adjusted such as when both the confidence level and the severity indicator value are at their lowest value. For example, the least aggressive response can be initiated, selected, or adjusted such as when the confidence level is moderate and the severity indicator value is low. In such an example, it can be desirable to respond using the least aggressive response even though the confidence level is not low because of the low severity indicator value of the myocardial ischemia event.

In an example, as in the example of FIG. 5, the moderately aggressive response can be initiated, selected, or adjusted such as when the confidence level is moderate and the severity indicator value is moderate.

There exist many possible relationships between the severity indicator value, the confidence level, and the graded response, and the example of FIG. 5 is but one possible relationship.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code can be tangibly stored on one or more volatile or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An apparatus comprising:
an ambulatory medical device, including a ischemia detector circuit configured to detect an indication of a myocardial ischemia event; and
a processor circuit, configured to be communicatively coupled to the ischemia detector and to:
receive the indication of the myocardial ischemia event;
determine a confidence level of the myocardial ischemia event having occurred; and
respond using the confidence level, the responding including at least one of initiating, selecting, or adjusting a response.

2. The apparatus of claim 1, wherein the processor circuit is configured to determine a severity indicator value of the indication of the myocardial ischemia event.

3. The apparatus of claim 2, wherein the processor circuit is configured such that the responding comprises responding using both the severity indicator value and the confidence level.

4. The apparatus of claim 3, wherein the processor circuit is configured such that the severity indicator is multi-valued and the confidence level is multi-valued.

5. The apparatus of claim 1, wherein the processor circuit is configured such that the determining the confidence level comprises using a regression model.

6. The apparatus of claim 5, wherein the processor circuit is configured such that:
the detecting the indication of the myocardial ischemia event comprises using one or more sensor measurements from the ischemia detector circuit to detect the indication of the myocardial ischemia event; and
the determining the confidence level comprises computing a probability according to:
$$P = \frac{1}{1 + {e}^{- z}} ,$$
wherein z=b₀+b₁X₁+b₂X₂+ . . . +bₘXₘ, P is a probability of a myocardial ischemia event occurring, z is a measure of a total contribution of all of the one or more sensor measurements used, b₀ is a logistic regression intercept, and b₁, b₂, . . . bₘ are the logistic regression coefficients of the one or more sensor measurements X₁, X₂, . . . Xₘ respectively.

7. The apparatus of claim 1, wherein the ambulatory medical device comprises an implantable medical device; and wherein the implantable medical device includes the processor circuit.

8. The apparatus of claim 1, wherein the processor circuit is configured such that the confidence level is determined using a time-wise sequence of multiple indications of the myocardial ischemia event.

9. A device-readable medium including instructions that, when performed by the device, comprise:
detecting an indication of a myocardial ischemia event;
determining a confidence level of the myocardial ischemia event having occurred; and
responding using the confidence level, the responding including at least one of initiating, selecting, or adjusting a response.

10. The device-readable medium of claim 9, wherein the instructions that, when performed by the device, comprise determining a severity indicator value of the indication of the myocardial ischemia event.

11. The device-readable medium of claim 10, wherein the responding comprises responding using both the severity indicator value and the confidence level.

12. The device-readable medium of claim 11, wherein the severity indicator is multi-valued and the confidence level is multi-valued.

13. The device-readable medium of claim 9, wherein the determining the confidence level comprises using a regression model.

14. The device-readable medium of claim 13, wherein:
the detecting the indication of the myocardial ischemia event comprises using one or more sensor measurements to detect the indication of the myocardial ischemia event; and
the determining the confidence level comprises computing a probability according to:
$$P = \frac{1}{1 + {e}^{- z}} ,$$
wherein z=b₀+b₁X₁+b₂X₂+ . . . +bₘXₘ, P is a probability of a myocardial ischemia event occurring, z is a measure of a total contribution of all of the one or more sensor measurements used, b₀ is a logistic regression intercept, and b₁, b₂, . . . bₘ are the logistic regression coefficients of the one or more sensor measurements X₁, X₂, . . . Xₘ respectively.

15. The device-readable medium of claim 9, wherein the responding comprises providing a local alert.

16. The device-readable medium of claim 9, wherein the determining the confidence level comprises using a time-wise sequence of multiple indications of the myocardial ischemia event.

17. A method comprising:
detecting an indication of a myocardial ischemia event using an ambulatory medical device;
determining a confidence level of the myocardial ischemia event having occurred; and
responding using the confidence level, the responding including at least one of initiating, selecting, or adjusting a response.

18. The method of claim 17, comprising determining a severity indicator value of the indication of the myocardial ischemia event.

19. The method of claim 18, wherein the responding comprises responding using both the severity indicator value and the confidence level.

20. The method of claim 19, wherein the severity indicator is multi-valued and the confidence level is multi-valued.
